(19) United States
(12) Patent Application Publication
(10) Pub. No.: US 2021/0174907 A1
(43) Pub. Date: Jun. 10, 2021

(54) METHOD OF MACHINE LEARNING, EMPLOYING BAYESIAN LATENT CLASS INFERENCE: COMBINING MULTIPLE GENOMIC FEATURE DETECTION ALGORITHMS TO PRODUCE AN INTEGRATED GENOMIC FEATURE SET WITH SPECIFICITY, SENSITIVITY AND ACCURACY

(71) Applicant: GENFORMATIC LLC, AUSTIN, TX (US)

(72) Inventors: Aaron J. MACKEY, Amherst, VA (US); Brandi CANTAREL, Austin, TX (US); Justin REESE, Austin, TX (US); Daniel B. WEAVER, Austin, TX (US)

(21) Appl. No.: 16/926,468

(22) Filed: Jul. 10, 2020

Related U.S. Application Data
(63) Continuation of application No. 14/083,356, filed on Nov. 18, 2013, now abandoned.
(60) Provisional application No. 61/727,655, filed on Nov. 16, 2012.

(51) Int. Cl. G16B 40/00; G06N 7/00

(52) U.S. Cl. CPC G16B 40/00; G16B 20/00; G06N 7/005

(57) ABSTRACT

BAYSIC (BAYesian System for Integrated Combination) combines sets of genomic and other biological data features to optimize selected data feature attributes, for example, detecting genome variants including single nucleotide variants (SNVs) and small insertion/deletions in genomes. The present disclosure presents one possible embodiment employing BAYSIC to combine single nucleotide variants detected by several distinct variant calling methods into an integrated SNV call set that is more accurate than any single SNV calling method or any ad hoc method of combining call sets. BAYSIC is a, tested and validated method using unsupervised machine learning, employing Bayesian latent class inference to combine variant sets produced by different packages.

### CROSS-REFERENCE TO RELATED APPLICATION

This application claims the benefit of U.S. Provisional Application No. 61/727,655, filed Nov. 16, 2012, the contents of which are incorporated by reference in their entirety.

### FIELD

The present disclosure relates to one or more methods and apparatuses for genomic feature detection and applications of that technology in biomedical research, clinical research, clinical trials and clinical medicine, especially oncology, in vitro fertilization, genetic disease diagnosis, disease risk prediction and pharmacogenomics and drug efficacy and risk evaluation.

### BACKGROUND

The advent of the genomic era and the generation of large databases of genomic sequence information have transformed many aspects of biological and medical science. Biology, genetics, and medicine have embraced the large volumes of genomic data that have accumulated and efforts to discover new knowledge by analyzing genomic data have transformed biomedical research and will soon transform clinical medicine into more computationally intense disciplines, reliant upon large databases containing huge amount of genomic and other biological and medical information. Substantial funding for development of bioinformatic tools and computational analysis methods to translate genome sequence information into data with analytical validity and clinical utility were fueled by the huge public and private investments that funded the human genome project. Additional genome projects in other organisms and followup efforts spawned by the human genome project also funded continued computational tools and bioinformatic methods development. One thousand genomes, the HapMap project and tremendous numbers of Genome Wide Association Studies also added to the arsenal of tools and methods available to analyze genome sequence data, and other genomic, transcriptomic, proteomic, metabolomic and systems biology information. See, e.g., McKenna A, Hanna M, Banks E, Sivachenko A, Cibulskis K, Kernytsky A, Garimella K, Altshuler D, Gabriel S, Daly M et al: The Genome Analysis Toolkit: a MapReduce framework for analyzing next-generation DNA sequencing data. Genome Res 2010, 20(9):1297-1303; Challis D, Yu J, Evani U S, Jackson A R, Paithankar S, Coarfa C, Milosavljevic A, Gibbs R A, Yu F: An integrative variant analysis suite for whole exome next-generation sequencing data. BMC Bioinformatics 2012, 13:8. E. G, G. M: Haplotype-based variant detection from short-read sequencing. arXivorg 2012, 1207.3907; Danecek P, Auton A, Abecasis G, Albers C A, Banks E, DePristo M A, Handsaker R E, Lunter G, Marth G T, Sherry S T et al: The variant call format and VCFtools. Bioinformatics 2011, 27(15):2156-2158; Forbes S A, Bindal N, Bamford S, Cole C, Kok C Y, Beare D, Jia M, Shepherd R, Leung K, Menzies A et al: COSMIC: mining complete cancer genomes in the Catalogue of Somatic Mutations in Cancer. Nucleic Acids Res 2011, 39(Database issue):D945-950.

However, despite great effort at developing accurate methods to discover and detect genomic sequence or genotype differences, the current state of the art is far less than perfect. To survey the genome sequence differences that distinguish two groups, one healthy and the other sick, it is obviously of fundamental importance to minimize false positive and false negative genome sequence differences. Likewise, methods to reliably detect sequence differences that differentiate diseased and healthy tissues from the same individual are essential if the characteristic mutations that reveal disease prognosis or response to treatment are to be discovered, much less become clinically actionable. Various methods that have been developed to address these detection problems often disagree, emphasizing the inherent problem of discriminating real sequence differences against the background of sequencing artifacts and other spurious noise. The consequent problem of accurate variant detection and the related detection error tradeoff conundrum—where increased sensitivity reduces specificity and enhanced specificity diminishes sensitivity—pose challenges that potentially impair the reliability and clinical utility of genome sequence information.

The present invention and disclosure present a solution to this important genomic feature detection problem, and enables embodiments that significantly reduce the detection error tradeoff problem in a formal probabilistic framework, allowing the user to find an optimal solution that simultaneously enhances specificity and sensitivity of genomic feature detection data, but also permits the user to tune the method to minimize false negative rates or false positive rates, as the particular application demands. Moreover, this invention extends beyond the specific problem of genomic variant detection and should be recognized as a general solution to the difficult and important problem of combining the outputs from different methods of genomic feature detection, while preserving the most important advantages and minimizing the limitations of the various input feature detection methods so combined.

### BRIEF DESCRIPTION OF THE DRAWINGS

Implementations of the present technology will now be described, by way of example only, with reference to the attached figures, wherein:

FIG. 1 is Flowchart describing an example of BAYesian System for Integrated Combination (BAYSIC) algorithm for producing sets of single nucleotide variants (SNVs) with improved sensitivity and selectivity, according to the present disclosure.

FIG. 2 illustrates an example of an observed agreement amongst variant calling programs according to the present disclosure.

FIG. 3 illustrates observed sensitivity and specificity of variant calling programs and BAYSIC.

FIG. 4 illustrates observed sensitivity and specificity of variant calling programs and BAYSIC.

FIG. 5 illustrates detected somatic mutations that were present in COSMIC using variant calling programs and BAYSIC.

### DETAILED DESCRIPTION

For simplicity and clarity of illustration, where appropriate, reference numerals have been repeated among the different figures to indicate corresponding or analogous elements. In addition, numerous specific details are set forth in order to provide a thorough understanding of the implementations described herein. However, those of ordinary skill in the art will understand that the implementations described herein can be practiced without these specific details. In other instances, methods, procedures and components have not been described in detail so as not to obscure the related relevant feature being described. Also, the description is not to be considered as limiting the scope of the implementations described herein.

Unless otherwise obvious from the context, the meaning of the terms below shall be as defined in this document, in addition to any commonly understood or dictionary definition of the term. “Genomic” or “genome” or “genome sequence” or “genomic sequence” or “genome data” or “genomic data”: consisting of, or pertaining to or relating to any of the following—DNA, RNA, nucleic acid sequences, nucleotide sequences, DNA sequences or RNA sequences, or DNA or RNA sequence data, genetic material of living organisms and any information contained therein, protein data, protein sequence data, trancriptome data or RNAseq data, genotype data, including but not limited to the output data from genome or transcriptome sequencing machines, instruments or devices, or genotyping machines, instruments, arrays, chips or devices. “Genomic feature” or “genomic data feature”: any identifiable genome or genomic or genotype sequence property or characteristic, including but not limited to any sequence or nucleotide change, alteration, substitution, transition, transversion, mutation, inversion, deletion, duplication, insertion, translocation, palindrome, base-pairing, alternative base pairing, three dimensional structure, three dimensional association, hairpin, secondary structure, sequence motif, sequence alignment, alternative sequence alignment, methylation, acetylation, or other base modification, signal, classifer, signature or any other distinguishing characteristic or alteration of any single or multi-base genome or genomic sequence data, or DNA or RNA nucleotide or base. “Genomic feature attribute” or “genomic data feature attribute”: any quality, condition, metric, quantifiable or qualitative characteristic, or other measurable property relating to, or exhibited by a genomic feature or genomic data feature.

A variety of analytic methods are employed to discover or detect features of interest in genomic, transcriptomic, proteomic, and other biological or medical data, including, but not limited to variants, polymorphisms, mutations or similar sequence or position-specific alterations in genomic, transcriptomic or proteomic data, in particular. The present disclosure presents a novel means of combining the emitted output of multiple algorithms that operate to detect a data feature, or the contents of databases that contain a data feature, or some combination of algorithmic output sets and database contents that detect or contain a data feature, to produce a single integrated data-feature set that optimizes selected data attributes, including but not limited to accuracy, precision, sensitivity, specificity, false-positive rate or false negative rate.

By way of illustration only, we describe at least one possible embodiment—namely, BAYSIC. BAYSIC is a machine learning method implementing a fully Bayesian latent class inference engine to produce an optimal set of genomic variant calls or somatic mutation calls. BAYSIC enables integration of multiple distinct and discordant genomic variant call sets produced by distinct variant detection algorithms into a single set of more accurate genomic variant calls with a user-specified posterior probability. BAYSIC operates completely without reference to, or need of, any “gold-standard” or “true-validated” data. Adjustment of BAYSIC's posterior probability threshold allows the user to tune BAYSIC, for instance, minimizing false-positive or false-negative error rates.

BAYSIC provides a convenient method for combining SNP calls from variant calling programs of the users choice to yield a high-confidence set of SNP calls with improved sensitivity and specificity over the SNP call sets provided as input. Further, BAYSIC allows the user to specify a posterior probability cutoff according to his/her needs. For applications for which sensitivity is a priority, this cutoff can be set low to minimize false negatives, and for applications for which specificity is a priority can be set high to minimize false positives.

The present disclosure includes at least one embodiment of BAYSIC including three applications, namely 1) improved germline genomic SNV calling in biomedical research, disease diagnosis, prognosis and therapy; 2) improved somatic SNV mutation detection, especially in cancer diagnosis, prognosis and care, and other clinical medicine contexts; and 3) improved structural variant detection for genetic disease diagnosis and disease risk estimation. Additionally, we have provided other applications within the present disclosure. Also, the present disclosure describes some applications in contexts other than genomic variant and somatic mutation detection.

Applications of Genome Sequence Analysis, Including but not Limit to Biological Research, Medical Research, Translational Medicine, Clinical Trials and Clinical Treatment

The falling cost of next generation sequencing makes it feasible for biomedical research scientists and clinicians to implement genome and exome sequencing to advance research discovery, and provide diagnostic, prognostic and therapeutic insights in clinical medicine. However, the potential uses of genomic data depend fundamentally upon accurate genomic variant detection. Without maximally sensitive and specific genomic variant discovery or detection, the analytical validity and clinical utility of genomic data can be compromised. Importantly, the presently described variant calling method combines output from multiple variant calling software tools, and mathematically optimizes sensitivity and specificity using Bayesian inference and machine learning. The BAYSIC variant identification system can simultaneously minimize false positives and false negatives, detecting variants with unmatched precision and accuracy.

Using Genomic Data Analysis to Accelerate Research and Improve Clinical Care

A physician can use patient genome sequence or genotype data to predict cancer predisposition—for instance, using established correlations between genomic variants and higher or lower relative risks of cancer to forecast future cancer risk based upon the presence or absence of those risk alleles in a patient's genome. Alternatively, an oncologist can use a patient's genome sequence data to design personalized treatment protocols. For example, detecting variants known to be associated with rapid disease progression and poorer prognosis, or efficacy of new therapies would provide actionable insight to a physician, allowing her to move the patient immediately into an alternative treatment regimen. Likewise, genomic data can reveal the presence of genomic variants that are associated with heightened or reduced efficacy for particular chemotherapeutic agents. Armed with more complete and accurate knowledge of the actual genomic variation present in a patient's tumor, therapy can be modified to use drugs selected for maximum efficacy and safety and avoid therapy that may only inflict only pain and needless suffering.

Using Genomic Data Analysis to Advance Cancer Research

Genomic data analysis can also be used to accelerate cancer research, including retrospective or prospective association studies to discover new correlations between genomic markers and patient or tumor phenotype. Some genomic markers have known associations with malignant tissue drug sensitivity. Similarly, genomic analysis can inform clinical trials to test patient responses to new drugs and validate companion diagnostic tests for new drugs. Companion diagnostic tests stratify patient populations into those patients more or less likely to respond to treatment, or into patient groups for which treatment can be safe and those for whom treatment can post unacceptable risks. It is now feasible to do genome or exome wide association studies with improved power to detect variants of small effect, or explore epistatic interactions among mutations or examine possible epigenetic correlates of cancer risk, progression and survival. Further, declining sequencing costs will allow large cancer centers to enroll growing numbers of patients in sequencing studies. The ensuing data surge, however, and the concomitant increase in analytical complexity and data management challenges will be problematic. As the scope and pace of genomic research intensifies, advanced computational approaches to genomic data analysis will yield new insights. Translating the insights of cancer genomics into novel therapeutic interventions and improved remission rates and survival are the ultimate objective.

Sequencing and Analyzing Tumor-Normal Pairs

An example protocol for using genome sequencing in research or clinical oncology is to sequence tumor-normal sample pairs. Sequencing tumor/normal pairs enables comparison of the genome sequence of healthy tissue to the genome sequence of cancerous tissue. Sequence variants detected in neoplasms but not present in normal somatic tissue can be mutations with implications for: a) forecasting disease risk; b) providing early disease diagnosis; c) predicting the probable course of disease progression; d) improving treatment efficacy and safety; and, e) improving patient outcomes and survival.

The differences between the normal and tumor genomes represent somatic mutations particular to the cancerous cells, which can be used to investigate the cause of the cancer, or used in retrospective or prospective studies involving thousands or tens of thousands of patients to evaluate potential associations between the detected variant and the variable of interest; e.g., response to treatment or drug efficacy. This strategy of using a subject as their own control reduces noise considerably compared with a strategy of comparing subjects to a reference sequence (for which phenotype data is often not available).

1) BAYSIC (Bayesian System for Integrating Calls)

BAYSIC Algorithm

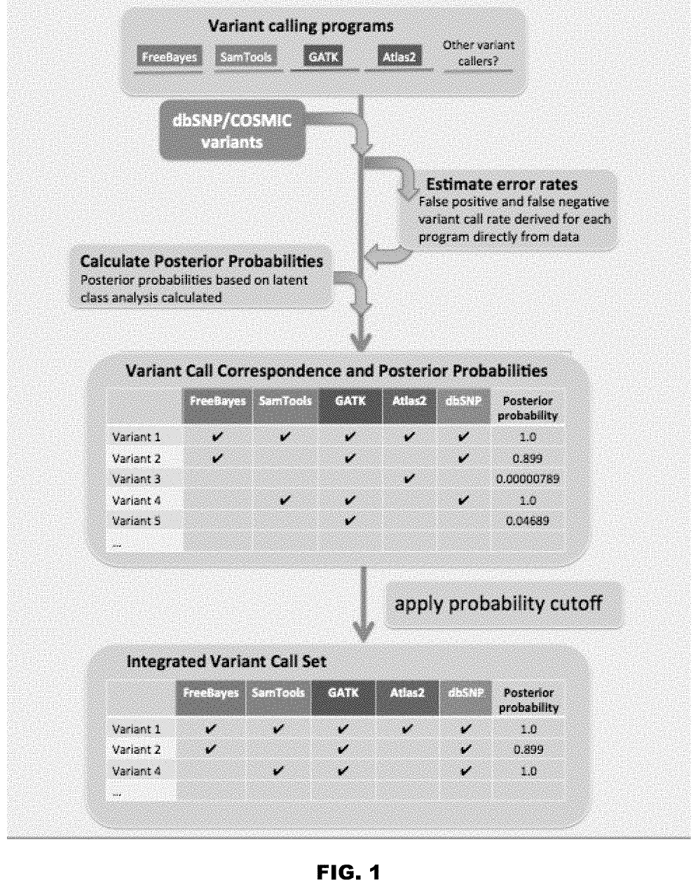

BAYSIC is a method combining sets of SNVs detected by one more existing programs into an integrated set of variants with improved sensitivity and specificity (See FIG. 1). The user provides variant calls from one or more variant calling programs of their choice in VCF format and a posterior probability cutoff. dbSNP information may be included as an additional source of variant information. For each type of error rate to be estimated (e.g., false positive or false negative), BAYSIC selects random values from a beta distribution with shape parameters a of 1 and b of 2 for many (tens of thousands of Hidden Markov Chain Monte Carlo iterations; here 120,000 iterations) to yield an estimated error rate. Posterior probability for each possible combination of agreement amongst variant calling programs and dbSNP are calculated as:

$$\frac{θ {{\prod }^{r}}_{i = 1} {{β}_{i}^{1 - {ϰ}_{i}} ( 1 - {β}_{i} )}^{{ϰ}_{i}}}{θ {{\prod }^{r}}_{i = 1} {{β}_{i}^{1 - {ϰ}_{i}} ( 1 - {β}_{i} )}^{{ϰ}_{i}} + ( 1 - θ ) {{\prod }^{r}}_{i = 1} {{α}_{i}^{{ϰ}_{i}} ( 1 - {α}_{i} )}^{1 - {ϰ}_{i}}}$$

where r is the number of variant calling programs used, a, is the false positive rate for the i^{th} program, βᵢ is the false negative rate for the i^{th} program, and 0 is the estimate of rate of overall SNP occurrence, xᵢ is 0 or 1 depending on whether the i^{th} variant calling program called a SNP at the given location. For each variant, a posterior probability is determined based on the programs which called the variant, and the posterior probability cutoff is applied to yield an integrated variant call set.

FIG. 1 is Flowchart describing the BAYSIC algorithm for producing sets of SNV with improved sensitivity and selectivity.

BAYSIC combines variant call sets produced by variant calling programs into a set of high-confidence variant calls. BAYSIC uses a Bayesian statistical method to combine output from 1 or more variant calling programs, or output from calling methods and the contents of a database of SNVs—e.g., dbSNP (FIG. 1). The user provides output from each variant calling program in VCF format as well as a desired posterior probability cutoff, based on the user's tolerance for false positive and false negative SNP calls.

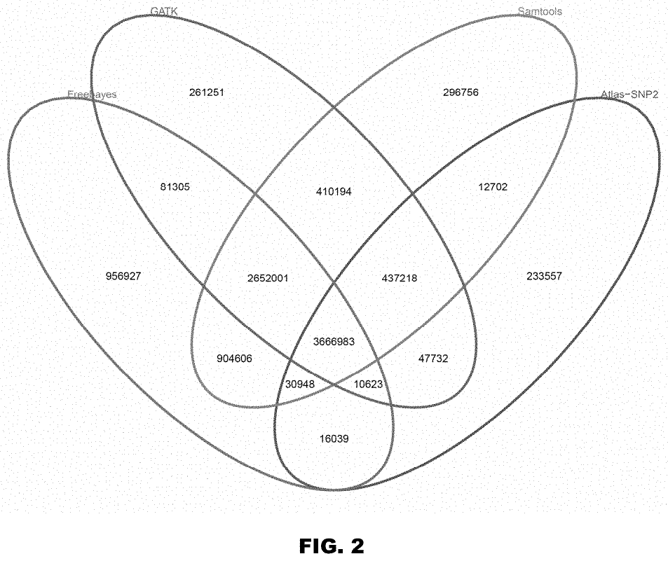

In an example study, BAYSIC analyzed Single Nucleotide Variants and small insertions and deletions (collectively, hereafter “SNVs”) predicted from standard BAM files using Samtools, GATK, FreeBayes and Atlas2. The intersection and union of the SNVs predicted by all callers or any of them was also determined. Note that the union of calls by any method is an upper bound on sensitivity, while the intersection of calls by all methods represents the specificity limit. (See FIG. 2).

The sensitivity of the Bayesian optimization method was calculated by comparing the SNV predictions to genotypes determined on an orthogonal platform—a SNV array chip—and the percentage of real SNPs discovered with each caller was determined. Specificity was empirically determined employing the ratio of transitions to transversions as a proxy; human exomes average a Ts/Tv ratio of 2.8-3.0; whereas the Ts/Tv rate of non-CDS regions average 2.0-2.1.

Using the results of three different SNV prediction methods, and orthogonal SNV calls from chip genotype data, a generalized method is offered, producing an optimal classifier (BAYSIC method) that allows the user to obtain SNV calls more sensitive and specific than any single method. Posterior probabilities of the correct result for BAYSIC calls were obtained. Critically, no single method provides calls as specific and sensitive as BAYSIC.

FIG. 2 illustrates observed agreement amongst variant calling programs. Variants were called using FreeBayes, SamTools, GATK, and Atlas2. Agreement amongst the variant calling programs was determined based on variant position. Numbers of SNP variants called by the programs indicated by the enclosing ellipses is shown.

The alarmingly poor concordance among the SNV calling methods is evident. Many SNPs were present only in one set (296,756; 956,927; 233,557; 261,251 for SNP detected only by SamTools, FreeBayes, Atlas and GATK, respectively) (FIG. 2). Further, only 36.8% (3,666,983) of calls were present in all four sets, and only 82.5% (8,222,619) of SNPs were present in two or more sets. The obvious adverse clinical consequences of reliance upon incorrect SNV identification (for example O'Rawe J, Jiang T, Sun G, Wu Y, Wang W, Hu J, Bodily P, Tian L, Hakonarson H, Johnson W E et al: Low concordance of multiple variant-calling pipelines: practical implications for exome and genome sequencing. Genome medicine 2013, 5(3):28, which is hereby incorporated by reference) provide motivation for BAYSIC and illustrate the practical importance and potential aplications of this novel method for integrating SNV calls. BAYSIC allows users to combine two or more sets of genome variants. The user supplies one or more VCF files containing the sets to be combined and a posterior probability cutoff based on the user's tolerance for false positive and false negative errors (FIG. 1). Optionally, the user may also supply a set of known variants from third party databases in order to increase accuracy, such as dbSNP or COSMIC. The rate of false positive and false negative errors for each set of variant calls are estimated based on the input data using a MCMC simulation, and the posterior probability for each possible combination of agreement between the sets of calls is determined (see Methods). The posterior probability cutoff specified by the user can then applied, and each variant that passes the cutoff can be written out to a new VCF file containing the integrated set of variant calls.

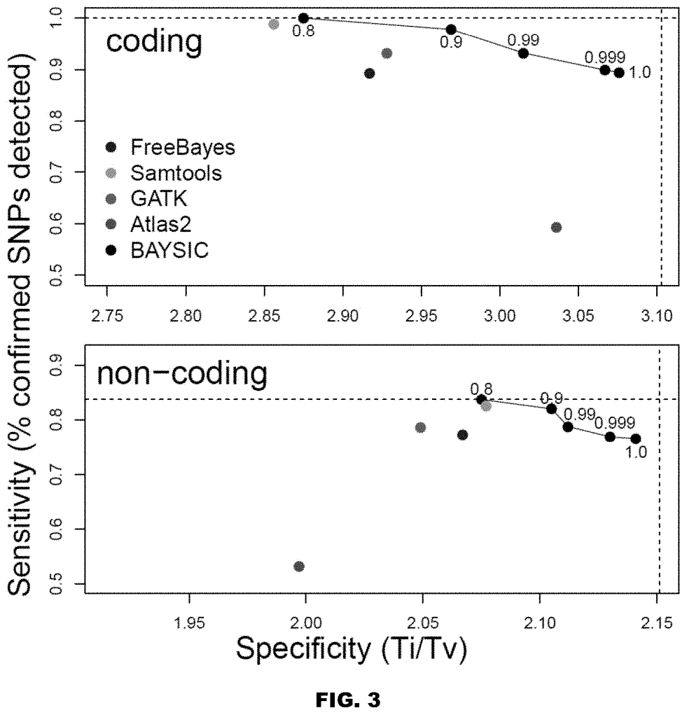

FIG. 3 illustrates observed sensitivity and specificity of variant calling programs and BAYSIC. Sensitivity of variant calling programs was measured by percent of SNPs confirmed by SNP-chip called by the given program. Selectivity was measured by transition/transversion ratio (Ti/Tv) of all SNP variants called by the given program. The sensitivity and specificity for SNPs in coding regions (top) and non-coding regions (bottom) is shown.

Additionally, sensitivity and specificity of both the union and intersection of the set of SNPs called by FreeBayes, SamTools and GATK was also measured (FIG. 3, dotted lines parallel to axes).

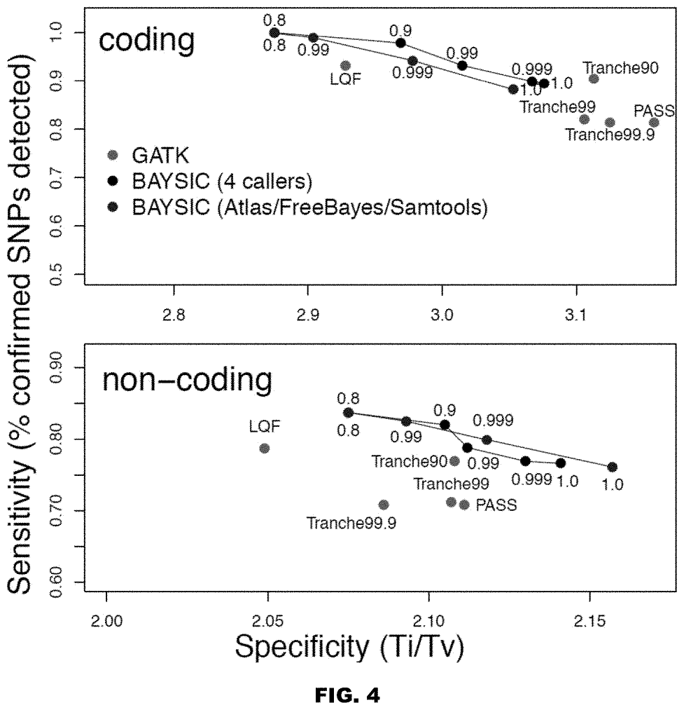

The sensitivity and specificity of BAYSIC produced with a range of posterior probability cutoffs, (from 0.8-1.0) when considering SNPs occurring in coding regions and noncoding regions was superior to SNV calls sets from FreeBayes, SamTools, GATK and Atlas2 (FIG. 3, top). When considering SNP calls occurring in non-coding regions, BAYSIC also performs impressively, producing a set of SNP calls with sensitivity and specificity greater than any set obtained by single SNV calling methods (FIGS. 3 and 4).

The advantages of the presently presented BAYSIC system are several. First, the BAYSIC calls have unprecedented sensitivity and specificity. The set of SNVs detected by BAYSIC are almost as sensitive as the union of all calls (the set of SNPS detected by any single included method—necessarily the most sensitive set), and simultaneously, nearly as specific as the intersection of all calls (the set defined by only those SNPs called by every incorporated method—necessarily the most specific set). There is usually a tradeoff between sensitivity and specificity—detectors with high sensitivity (few misses) sacrifice specificity (more false alarms). BAYSIC: optimizes this tradeoff to produce greater overall accuracy and precision than other methods.

Second, any combination of methods to detect SNVs can be incorporated as input to BAYSIC. BAYSIC represents a modular optimization of multiple independent SNV detection tools—any combination of multiple methods can be incorporated as input to BAYSIC. Consequently, as new variant calling methods are developed, those methods can be incorporated in BAYSIC. Allowing substitution of superior individual SNV detection methods (or other variant detectors) will improve overall performance, but the BAYSIC system will continue to produce the optimal result. The choice of posterior probability value for the BAYSIC system enables tuning the performance of BAYSIC to emphasize sensitivity or specificity as the particular application can demand.

Third, the choice of posterior probability value for the BAYSIC system enables tuning the performance of BAYSIC to emphasize sensitivity or specificity as the particular application demands. For example, in some clinical research applications, sensitivity can be maximized to produce candidate SNVs that will be validated and investigated with downstream analysis. In these cases, a user can apply a less stringest posterior probability cutoff to maximize sensitivity. Conversely, maximum selectivity is critical for many clinical applications in which downstream analysis is not feasible or desirable. In these cases, a user can apply a more stringent posterior probability to maximize specificity.

The BAYSIC method is applicable in wide range of contexts, and the general Bayesian inference of latent data feature classes should prove useful and offer advantages in contexts other than “simple” SNV calling. In particular, The BAYSIC system can be of value in cancer research and clinical care.

Development of New Enhancements of BAYSIC Optimized for Genome Analysis in Cancer

2) BAYSIC-NORMALIGNANT (BAYSIC Normal/Malignant)

The present disclosure has important applications in cancer research, and can be employed for the detection of somatic mutation in tumor/normal tissue pairs. Calling SNVs in sequence data from tumor-normal sample pairs should be simplified by the common origin of the samples—both arising from a single individual's genome. The signal to noise ratio of somatic mutations arising in cancer is thereby amplified. Nonetheless, calling SNVs in cancer samples can be challenging, because the sequence data can represent a heterogeneous mixture of normal and cancerous cells with different genomic signatures. Distinguishing the signal of an allele change in the malignant cells (e.g., AT>TT in cancer), from the background “noise” of the heterozygous normal state+sequencing error, can be a difficult problem. Further complications can arise from clonal expansions of distinct cancer cell lineages with diverse mutational spectra, copy number variants and ploidy changes.

Accurately assessing variants in tumor/normal samples or heterogeneous cell populations represent additional applications of the BAYSIC method.

The problem can be considered as analogous to variant calling, but it is necessary to account for more than the “called” allele at every position in the normal tissue in order to optimally assess the likelihood that the same or a different allele is present in the tumor. Additionally, tracking the average allele count across genomic segments can be informative of the copy number status of that segment. Copy number variation is a well characterized variant class often associated with cancer. One can discern the ploidy of the tumor genome as well, summing read depth across multiple segments or even chromosomes. Thus, optimization of variant calling in tumor/normal samples will require, at a minimum, consideration of the read depths or number of reads that support the called alleles at every position.

Consider the following example—for purposes of simplicity, copy number variation and ploidy analysis will be omitted from consideration, though it will be apparent how the analysis can be generalized to include determination of copy number and/or ploidy status. Assume that 8 of 100 reads from “normal” genome sequenced to 100× coverage show an A allele; and 92 reads show a T allele at that same position. Calling the SNP at the first locus using typical algorithms would likely produce a T/T genotype. Further suppose that histopathology or microscopic examination reveals that roughly 20% of cells show precancerous morphology. If the only information stored is the T/T genotype, then useful information will be discarded. For illustrative purposes, assume a second sample is sequenced (possibly from a subsequent sample that is part of a time series from the same tissue), and this sample produces 19 reads with an A allele versus 81 reads with T allele. Again, microscopy or histopathology indicates a pre-neoplastic morphology with ˜⅕ of cells displaying aberrations consistent with a precancerous condition. Selecting the “correct” call from the sequence data using standard procedures might once more suggest a T/T homozygote for the position. Assuming further, a third sample from later in time or from an adjacent slice of tissue yields 57 reads with T in the relevant position and 43 reads with an A and visual examination suggests that the sample is clearly cancerous. Perhaps for the first time, a variant call at the relevant position using standard variant calling software would produce a heterozygous A/T call.

One possible explanation of this distribution of alleles and the changing pattern over time is that the A allele can be an early diagnostic marker of transformation from benign to malignant phenotype. If only called variants are recorded from the sequence data of the various samples, those calls would fail to reveal the dynamic continuous allele frequency distribution and instead only record a single discrete change at a single sample and time point. Clearly the biology is more complex than a sudden switch in allele at a single time point. More importantly, the potential diagnostic insights are potentially far greater if the read depth and alignment evidence supporting the variant calls are used as relevant parameters or conditional probabilities.

Employing a Bayesian inference method at the outset, in contrast to a more standard variant calling tool, would produce an exploration of the relevant joint probability distribution and conditional dependencies, and would likely suggest that ˜20% of cells with a heterozygous genotype at the relevant position (˜20% A/T; ˜80% T/T) would produce a signal consistent with the observed pattern—(8=A vs. 92=T). Likewise, detailed exploration of the probability distribution landscape consistent with the sequencing data of 19 reads=A and 81 reads=T should produce alternative possibilities of ˜40% heterozygous A/T and ˜60% homozygous T; or 20% homozygous A and 80% homozygous T; and other options in between. Critically, the co-variation of the allele frequency with morphological phenotype can be treated as another parameter upon which posterior probabilities can be conditioned, and the model further elaborated to enhance its informative power.

In addition to implementation of BAYSIC′ which evaluates various values for α_{1 . . . n} (false positive calls), β_{1 . . . n} (false negative calls), and θ_{1 . . . n}, (probability of variant) at each variant position (n_{1 . . . j})), and for every method (Y_{1 . . . k}) to produce optimal variant calls conditioned on the evidence, the present technology also enables extension of the method—BAYSIC NORMALIGN—that implements a modified Gibbs sampling procedure (e.g., a Markov chain Monte Carlo process with simulated annealing) to explore the joint probability distribution (or conditional distribution) of various hyper-parameters, including base qualities, alignment scores, read depths, as well as cancer/normal cell mixture ratios, and other pertinent variables to produce a posterior probability that optimally identifies variation in tumor/normal sample pairs conditioned on the hyper-parameter evidence.

Using BAYSIC to Combine Sets of Somatic Mutation Calls Produced with Tumor/Normal Pair Data

A common application of genome sequencing is to sequence samples taken from normal and tumorous tissue and detect somatic mutations that may be involved in cancer. Many programs exist to detect somatic mutations, and the problem of combining these sets of somatic mutations is analogous to the problem of combining disparate sets of SNPs produced by different SNP detection programs.

We applied BAYSIC to this related problem of combining disparate sets of somatic mutation calls. Using sequencing data from tumor and normal pair from a single patient, we produced somatic mutation calls using Caveman, JointSNVMix, Somatic Sniper and Strelka, and then combined these four sets of somatic mutation calls using BAYSIC with a default posterior probability cutoff of 0.8.

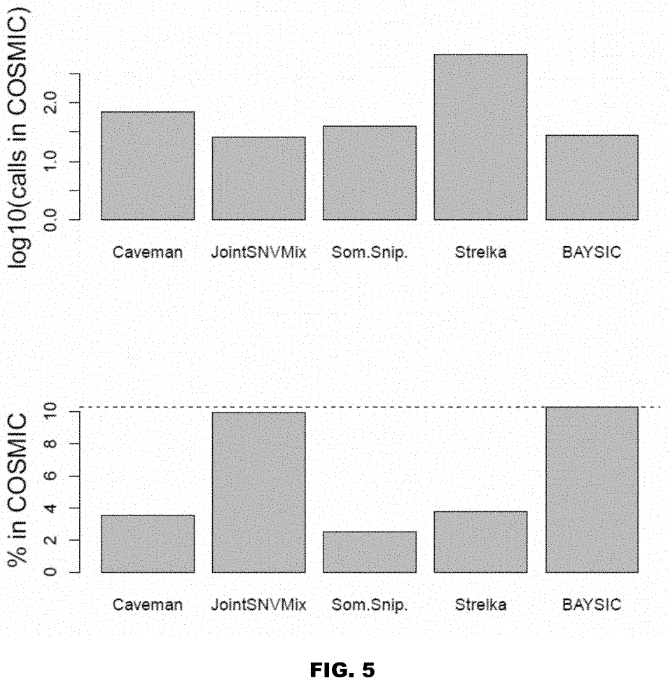

BAYSIC improved the specificity of the sets of somatic mutation calls used as input, as measured by the percent of somatic mutations present in COSMIC (a catalog of previously observed somatic mutations) (FIG. 5). As a measure of sensitivity, we measured the overall number of somatic mutations detected by each program that were present in COSMIC (a database of previously observed somatic mutations). Caveman, JointSNVMix, SomaticSniper, Strelka and BAYSIC detected 71, 26, 39, 651 and 28 somatic mutations that were present in COSMIC, respectively (FIG. 5). The sensitivity of BAYSIC, as measured by the overall number of somatic mutations detected by BAYSIC that were in COSMIC, was lower than the sets produced by all programs apart from JointSNVMix. Given the plethora of somatic mutation calls produced by most somatic mutation detection methods, the reduced complexity of the BAYSIC call set may provide advantages.

3) Baysic Structure

Importantly, it is now appreciated that structural variants (SVs) comprise a source of genomic variation that is particularly relevant in cancer. Moreover, it can be difficult, without implementation of the present technology, to accurately identify SVs without exhaustive, time-consuming and expensive validation of predicted structural rearrangements.

A Bayesian inference latent classification analysis can be used to optimally combine output from existing structural variant identification methods. The system will “learn”, creating posterior probabilities of correct structural variant calls conditioned on the evidence of performance of each method and the system in accurately characterizing known structural variant features in sequence data.

The present disclosure includes a method that can be completely analogous to the algorithmic foundation of BAYSIC, but modified to handle the more complex nature of structural rearrangements. BAYSIC structure will undoubtedly explore additional parameter space, as more variables will be needed to properly model the more complex nature of inversions, insertions, deletions, translocations, and the various nested forms of those structures that can be present in cancer genomes, to produce an optimal structural variant output.

4)—Other Applications

The present disclosure includes a method of Bayesian inference latent class analysis that can reasonably be applied to many other problems, including but not limited to biological and medical problems. It is common for many programs to be written to address biological problems and these programs frequently produce sets of data that have poor concordance with one another. Other embodiments of our Bayesian inference latent class analysis could be used to combine sets of data features emitted by these programs. Additional applications are too numerous to exhaustively elaborate, and include but are not limited to sets of predicted methylated nucleotide sites, sets of predicted promoter regions, miRNA target sites or other regions correlated with gene expression patterns, or sets of histone modification sites, drug safety, efficacy or drug interactions and their correlations with genomic data, disease vulnerability or medical condition predisposition correlations with genomic data, and other phenotype associations with genomic data, to name but a few.

Pseudo Code implementation of BAYSIC

```
# construct contingency table with list of variant callers that called a
variant at
# each position
for each variant call set
  for each variant
    mark variant caller as having called variant at position of current
variant
  end
end
for each variant caller
  for each parameter (false positive, false negative, and overall rate of
variant occurrence)
    estimate parameter using MCMC
# calculate posterior probability for each possible combination of variant
callers for each possible combination of variant caller
  posterior probability of variant for this combination of callers =
    calculate_posterior_probability(this combination of callers)
# write out combined variant set
cutoff posterior probability = user specified posterior probability ∥ 0.8
for each variant call set
  for each variant
    retrieve posterior probability for this variant based on which variant
callers detected variant
    if (posterior probability for this variant > cutoff posterior probability)
      output variant to file containing combined variant set
  end
end
subroutine calculate_posterior_probability(this combination of callers)
  posterior probability =
$\frac{θ {\prod }_{i = 1}^{r} {{β}_{i}^{1 - {x}_{i}} ( 1 - {β}_{i} )}^{{x}_{i}}}{θ {\prod }_{i = 1}^{r} {{β}_{i}^{1 - {x}_{i}} ( 1 - {β}_{i} )}^{{x}_{i}} + ( 1 - θ ) {\prod }_{i = 1}^{r} {{α}_{i}^{{x}_{i}} ( 1 - {α}_{i} )}^{1 - {x}_{i}}}$
```

where r is the number of variant calling programs used, cd is the false positive rate for the ith program, βi is the false negative rate for the ith program, and θ is the estimate of rate of overall SNP occurrence, xi is 0 or 1 depending on whether the ith variant calling program called a SNP at the given location.

What is claimed is:

1. A method comprising:
combining, at a processor, genomic feature detection data;
outputting the combined genomic feature data.

2. The method of claim 1, further comprising:
employing a Bayesian latent class inference engine in combining the genomic feature detection data.

3. The method of claim 1, further comprising:
employing unsupervised machine learning in combining the genomic feature detection data.

4. The method of claim 3, further comprising:
implementing a Bayesian latent class inference engine conducting the unsupervised machine learning in combining the genomic feature detection data.

5. The method of claim 4, further comprising:
generating an optimal genomic data feature detection combination, or an optimal genomic data feature detection output according to a selected data attribute.

6. The method of claim 4, further comprising:
substantially concomitantly, optimizing more than one genomic feature detection attribute.

7. The method of claim 6, further comprising:
assigning a probability of each genomic feature detection event detecting a true genomic data feature as a predetermined quantity with a range of zero to one.

8. The method of claim 6, further comprising:
assigning a probability of each genomic data attribute detection event detecting a true genomic data feature attribute as a predetermined quantity with a range of zero to one.

9. The method of claim 8, further comprising:
enabling tuning system or method operation to alter combining genomic feature detection data, or genomic feature attribute data, according to a selected probability quantity.

10. The method of claim 9, further comprising:
enabling tuning system or method operation to alter outputting combined genomic feature detection data, or genomic feature attribute data, according to a selected probability quantity.

11. The method of claim 10, further comprising:
enabling tuning system or method operation to alter system output to emphasize one or more genomic data feature attributes or one more system or method performance metrics.

12. The method of claim 11, wherein the one or more system performance metrics or data feature attributes includes at least one of enhancing sensitivity or specificity.

13. The method of claim 11, wherein the one or more system performance metrics or data feature attributes includes enhancing accuracy.

14. The method of claim 11, wherein the one or more system performance metrics or data feature attributes includes one of minimizing false positives or minimizing false negatives.

15. The method of claim 11, wherein the one or more system performance metrics or data feature attributes includes at least one of minimizing false positives or minimizing false negatives.

16. The method of claim 11, wherein the one or more system performance metrics or data feature attributes includes substantially concomitantly minimizing false negatives and false positives.

17. The method of claim 11, wherein the one or more system performance metrics or data feature attributes includes substantially concomitantly optimizing sensitivity and specificity.

18. The method of claim 17, further comprising:
detecting, at a processor, at least one correlation or association relating one genomic feature detection data to another genomic feature detection data, or relating one genomic feature data attribute to another genomic feature data attribute, or relating one genomic feature detection data to one genomic feature attribute data;
outputting the correlated or associated genomic feature detection data, genomic feature attribute data, or at least one combination of correlated or associated genomic feature detection data and genomic feature attribute data.

19. The method of claim 18, further comprising:
combining, at a processor, at least one of genomic feature detection data or genomic feature attribute data with at least one of:
genomic feature attribute data or genomic feature detection data;
correlated or associated genomic feature detection data;
correlated or associated genomic feature attribute data;
microRNA data;
microRNA target data;
transcription factor data;
transcription factor binding site data;
enhancer data;
promoter data;
RNA splicing data;
DNA methylation data
DNA modification data;
DNA packing and three dimensional conformation data;
RNA editing data;
Long noncoding RNA data;
Histone methylation data;
Histone acetylation data;
Protein binding data
Protein conformation and structure data;
Genetic data;
Pedigree data;
Medical history data;
Microbiome data;
Epidemiological data;
Vaccine data;
Chemical toxiclogy data;
Chemical library data;
phenotype data;
gene pathway data;
protein pathway data;
biochemical pathway data;
gene ontology data;
medical subject matter heading data
clinical medical data;
drug data;
pharmacologic data;
pharmacogenomic data;
metabolomic data;
genomic, transcriptomic or proteomic data;
organ data;
immunologic data;
biological systems data;
other species data;
outputting the combined data.
